(19) United States
(12) Patent Application Publication
(10) Pub. No.: US 2025/0099499 A1
(43) Pub. Date: Mar. 27, 2025

(54) ORGANIC-INORGANIC REACTIVE SCINTILLATORS FOR USE IN DISEASE TREATMENT

(71) Applicant: Mission Support and Test Services, LLC, Las Vegas, NV (US)

(72) Inventors: James R. Tinsley, North Las Vegas, NV (US); John Di Benedetto, Santa Barbara, CA (US)

(21) Appl. No.: 18/893,773

(22) Filed: Sep. 23, 2024

Related U.S. Application Data
(60) Provisional application No. 63/539,793, filed on Sep. 21, 2023.

(51) Int. Cl. A61K 33/00; A61K 9/50; A61K 31/80; A61K 35/15; A61K 41/00

(52) U.S. Cl. CPC A61K 33/00; A61K 9/5068; A61K 31/80; A61K 35/15; A61K 41/0057

(57) ABSTRACT

A system and method for reducing a number of cancerous cells in the body through use of a treatment package a mixture of scintillating organic silicon-based polymer and a nitric oxide (NO) release agent at least partially surrounded by a functional material that facilitates consumption of the treatment package by a macrophage. The macrophage travels to the tumor and is then radiated with X-ray radiation causing the scintillating polymer to emit photons at a first wavelength which strike and causing the release agent to release substance toxic to the cancer cells. The scintillating polymer may comprise polydimethylsiloxane. The release agents may comprise one or more of the following: iron, copper, CrONO, Ruthenium (phen)(NO)(Cl), and Roussin's black salt. The scintillating polymer is selected such that the emitted photon wavelength corresponds to a wavelength of maximum absorption by the release agent.

### 1. STATEMENT REGARDING FEDERAL RIGHTS

This invention mas made with government support under Contract Number NNSS Prime Contract DE-NA0003624 effective Dec. 1, 2017, updated to include modifications through 0177, and was awarded by the U.S. Department of Energy, National Nuclear Security Administration. The government has certain rights in the invention.

### 2. FIELD OF THE INVENTION

The invention relates to medical use of small or nanoparticle therapeutic treatments and in particular to use of scintillating polymers to assist in the release of therapeutic agents.

### 3. RELATED ART

A potent weapon for the destruction of cancerous tumors in the body has been photodynamic therapy using lasers where light can reach the cancer cells. One variant of this type of targeted therapy uses a reagent that generates toxic levels of nitric oxide (NO) in targeted cells. These nitric oxide-generating reagents are activated by optical photons. The required optical photons can be generated by up conversion nanoparticles, allowing deeper but not unrestricted access. With both direct and/or multiphoton excitation, optical photons must reach the cells, limiting the applicability against deep tumors.

In a typical delivery strategy, NO-generating reagents and light capturing nanoparticles are delivered into tumors in microspheres that are carried to the tumor by macrophages. The nanoparticles within the microspheres may be referred to as the treatment package, which is consumed by macrophages. A critical hurtle to the overall effectivity of the NO release is the transfer of energy from the optical energy source to the NO-generating reagent through opaque tissues or other light blocking features of the body.

Foundational work in the field of using nanoparticles or nanosphere to expose NO to tumors was performed by Peter C. Ford and outlined in the paper titled NIR-Triggered Release of Caged Nitric Oxide Using Upconverting Nanostructured Material, published in 2012 and available from WileyOnlineLibrary.com (Small 2012, 8, No. 24, 3800-3805), which is incorporated in its entirety herein. Also incorporated by reference in its entirety herein is the article titled Multi-photon Excitation in Uncaging the Small Molecule Bioregulator Nitric Oxide as published in Philosophical Transactions of The Royal Society, Series A, Mathematical, Physical, and Engineering Sciences Volume 371:20120129 (2013).

As shown in FIG. 1 and FIG. 2, a nanosphere 104 or other shape is defined by an inner NO (nitric oxide) producing material 116 encapsulated or caged within a scintillating outer shell 118. The material of the outer shell 118 is selected such that it emits generated light energy 112 into the inner NO producing material 116 in response to external light energy 108, such as from a laser or other near infrared or visible light spectrum light energy. The resulting NO is shown to be biologically harmful to cancer cells. To introduce the nanosphere into the body, the outer surface nanosphere is covered with a functional material 128 which facilitates acceptance of the nanosphere into a macrophage (such as by the macrophage consuming the nanosphere). The tumor seeking macrophage 140 or other mechanism/organism, is placed in the body, such as by injection, and is attracted to the tumor or other unwanted growth. In one embodiment the carrier 128 comprises a surfaced-modified microsphere which is consumed by macrophages 140, thereby causing the entering the macrophages. Within the body, the macrophages natural behavior is to travel to the tumor cells.

As accurately set forth in the paper, the use of upconverting nanoparticles (UCNPs) 104 facilitate NO uncaging (release) from a well characterized precursor by 980 nm irradiation from a simple NIR diode laser operating in the continuous mode. Lanthanide-doped UCNPs are proposed, since such material can upconvert continuous-wave NIR light into UV and visible wavelengths that can be harnessed to drive the photoreactions of compounds anchored to their surfaces. Among the commonly used UCNPs, Yb/Er co-doped NaYF4 nanocrystals are efficient NIR-to-visible up conversion materials, the hexagonal phase having higher efficiency than the cubic phase nanocrystals. Silica coated hexagonal NaYF4: Yb/Er@NaYF4 UCNPs with core/shell structures and positive outer surfaces and are used to trigger photochemical NO release from an iron nitrosyl complex. The use of iron/sulfur/-nitrosyl cluster Roussin's black salt anion Fe4S3 (NO) 7-(RBS, Na+salt) may be used as the NO precursor. The photolysis of RBS at UV/visible wavelengths gave NO release at biologically relevant concentrations suitable tumor size reduction.

One challenge presented by this groundbreaking research and technique is the limitations inherent in use of light 108 for activating scintillation (light emission) from scintillating outer shell 118. For example, many tumors are located sufficiently deep within the body, or at a location which prevents sufficient light energy from reaching the therapeutic nanosphere 104. This limits secondary photon 112 production which in turn limits NO production from the NO producing material 116.

A proposed solution to enable treatment of tumors located deep within the body or at locations at which laser light will not penetrate is use of X-ray energy to activate scintillation of the light emitting material within the treatment package. One strategy is to use an analogous layered approach of a macrophage-microsphere-nanoparticle-NO release agent in a layered concentric sphere structure which is exposed to X-Rays instead of optical photons.

However, this approach suffers from several drawbacks as well. It became clear that only a small fraction of the incident 5 MeV therapeutic X-Rays would directly scintillate the nanoparticles due to the interaction between the X-Rays and the microspheres. Also, most studies on these processes are performed using moderate energy X-Ray (100-150 KeV) where the photoelectric effect has much of the deposited X-Ray energy into the nanoparticles. In the case of therapeutic sources much of the deposited energy will go to the production of Compton electrons which will scatter into the material surrounding the higher density nanoparticles.

As a result, the prior art treatment packages suffer from poor absorption of the X-ray energy. This in turn translates to lower scintillation output, which results in reduced production of NO. Consequently, treatment with X-ray energy is less effective at cancer treatment. Therefore, there exists a need to expand the novel work set forth in the above-mentioned papers to locations which are not as readily exposed to prior art light sources and which overcome the drawbacks of the prior art.

### SUMMARY

To overcome the drawbacks of the prior art and provide additional benefits, disclosed is a method for killing or reducing a number of cancerous cells in the body comprising providing a treatment package having a matrix comprising a mixture of scintillating polymer and a nitric oxide (NO) release agent at least partially surrounded by a functional material. Then, providing treatment packages to macrophage for consumption by the macrophage causing the treatment package to be within the macrophage and presenting the macrophage to a human body. The human body has cancer cells living within the human body. After waiting a period of time for the macrophage to travel to the cancer cells, directing X-ray radiation to the locations of the cancer cells, such that the X-ray radiation cause the scintillating polymer to emit energy at a first wavelength. The energy at a first wavelength causes the NO release agent to release nitric oxide, which reduces the number of cancer cells. The scintillating polymer is an organic silicon-based polymer.

In one embodiment, the matrix is a microsphere and the cancer cells form a tumor. The scintillating polymer may comprise at least polydimethylsiloxane. The NO release agent may comprise one or more of the following: iron, copper, CrONO, Ruthenium (phen)(NO)(Cl), and Roussin's black salt.

The scintillating polymer may be selected such that the first wavelength of the energy emitted corresponds to an energy wavelength that maximizes production of NO from the NO release agent. In addition, the scintillating polymer and the NO release agent may be selected such that an output wavelength of the scintillating polymer aligns with an activation wavelength of the NO release agent. In one treatment environment, the X-ray radiation dose is less than 5 MeV.

Also disclosed is a pharmaceutical treatment package for the delivery of treatment to cancer tumor cells comprising a core and a functional coating. The core comprises a combination of scintillating polymer and a release agent. The scintillating polymer comprising a polymer which is configured to absorb X-ray energy and in response thereto, emit light energy at a first wavelength. The release agent is a material which absorbs the light energy at the first wavelength and emits a substance that is toxic to cancer tumor cells in response to light energy at the first wavelength. The functional coating on at least a portion of an exterior of the core, the functional coating configured to facilitate acceptance or entry of the core into a macrophage.

In one configuration the polymer is a silicon polymer matrix. In one embodiment the polymer is selected to have 5% or less absorption loss at the first wavelength, such that the first wavelength is the polymer emission wavelength. The achievement of a 5% or less absorption loss is due to an emissive shift of a pendant phenyl/napthal group. As discussed herein the release agent may emit nitric oxide. In one embodiment, the polymer comprises polydimethylsiloxane.

It is contemplated that the release agent is one or more of the following: inorganic CrONO, Ruthenium (phen)(NO)(Cl), and Roussin's black salt. In addition, the scintillating polymer may be an organic silicone polymer with a pendant group aromaticity with a silane backbone to establish gas permeability. It is also disclosed that the core may consist of a scintillating monomers comprising functionalized naphthalene and a release agent which is dispersed in a non-scintillating polymer.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates an exemplary treatment package capable of producing NO or other chemicals which are toxic to tumors.

FIG. 2 illustrates an exemplary treatment package within an exemplary carrier, such as a macrophage which is exposed to red laser light.

FIG. 3 illustrates an example arrangement of use of a scintillating polymer containing a NO releasing agent to generate NO.

FIG. 4A illustrates an example arrangement of a matrix formed from a scintillating polymer with NO releasing agents imbedded therein.

FIG. 4B illustrates an alternative arrangement formed by layers of scintillating polymer and NO releasing agents.

FIG. 5 illustrates an exemplary silicon polymer configuration of a phenyl/methyl silane co-block polymer.

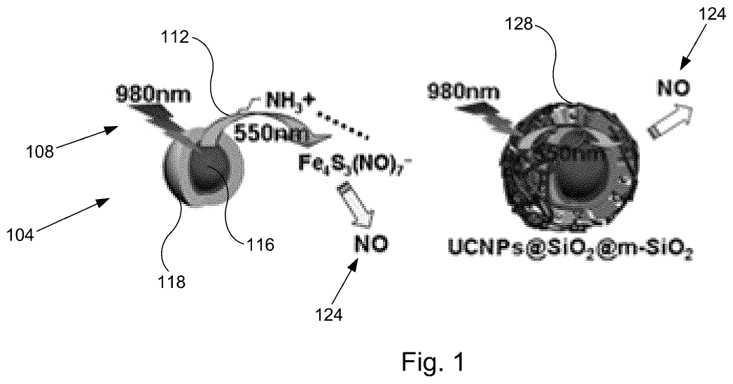

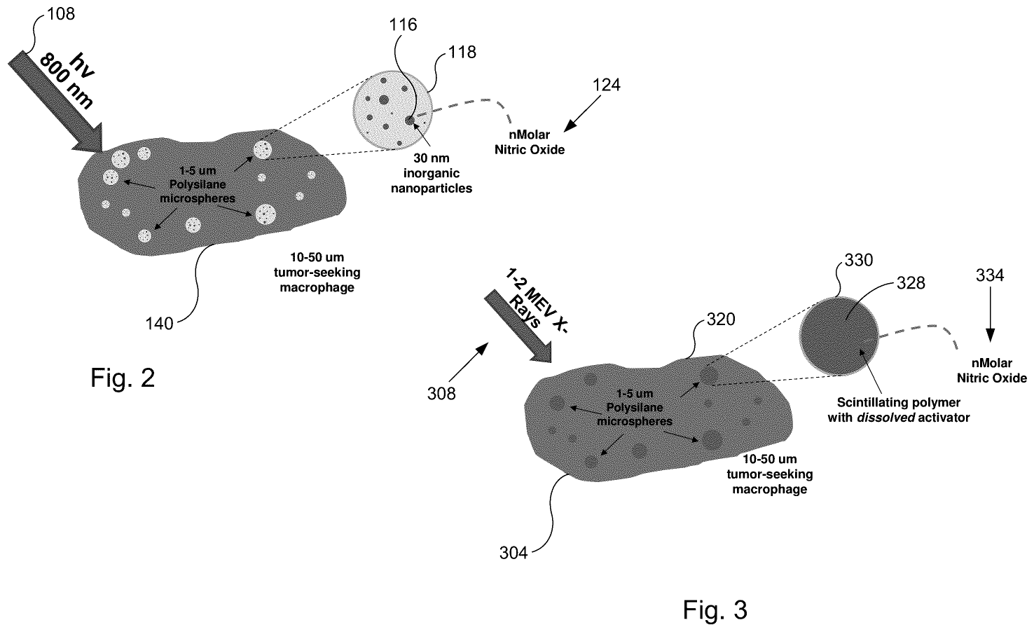

### DETAILED DESCRIPTION

To overcome the drawbacks of the prior art and provide additional benefits, disclosed is a treatment package which greatly increases nitric oxide (NO) production in response to X-ray exposure. This provides a success factor by improving the efficacy of delivery of X-Rays to the scintillating material and in turn increase NO release.

Disclosed herein is the use of organic scintillators as X-Ray capture agents with the NO release agent (such as but not limited to inorganic complexes such as CrONO, Ruthenium (phen)(NO)(Cl), and Roussin's black salt) dispersed throughout a matrix. Having the NO release agent dispersed throughout the matrix provides and improves the close contact between the nanoparticles and the NO release agents. In one variant, the matrix is a scintillating polymer, which may be arranged as a co-polymer. In another variant, scintillating monomers (such as functionalized naphthalene) and the NO-release agent, are dispersed in a non-scintillating polymer. A specific example of the organic component is a silicone polymer with pendant group aromaticity for enhanced scintillation yield with a silane backbone to provide maximum gas permeability. Although this innovation is described in terms of NO release, it is also disclosed that other cancer cell treatment agents may be selected for release by changing the release agent. In addition to or instead of the release agents which release NO, it is contemplated and disclosed that additional types of release agents may be in the matrix/polymer which forms the treatment package. Other release agents may be utilized. The released chemicals, other than NO, may include but are not limited to radical forming reagents such as peroxides, and other cytotoxic reagents.

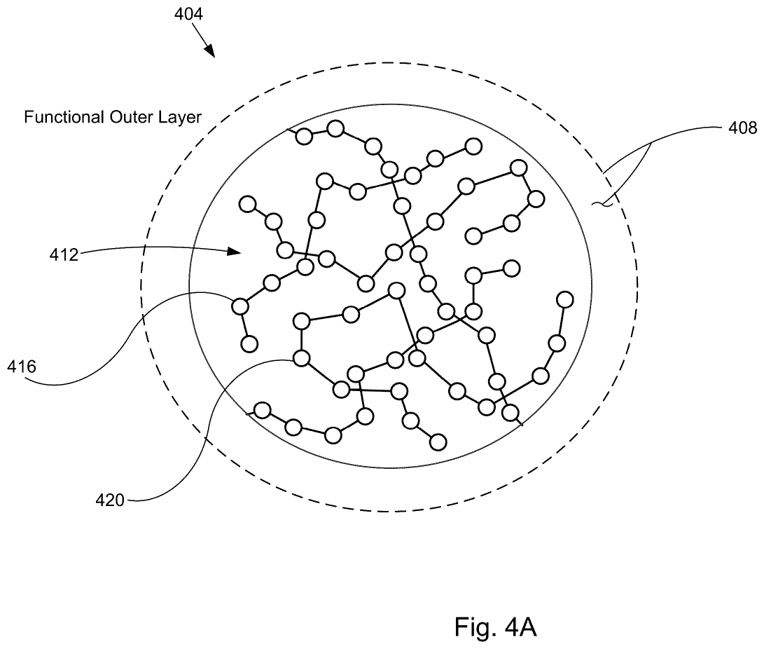

FIG. 3 illustrates an example arrangement of use of a scintillating polymer containing a NO releasing agent to generate NO. As shown in FIG. 3, macrophage 320 or other carrier suitable for transporting the treatment package 328 to cancer cells is provided such that the treatment package is a polymer created by a mix, layering, or other arrangement of scintillating material and NO release agents which emit NO 334. On the outside of the treatment package may be an optional protective coating 330 or the functional material which is consumable by the macrophage 320.

Once the treatment package 328 is inside the macrophage 320 and the macrophage is in or adjacent the tumor 360 or other unwanted growth/element within the body, the macrophage is exposed to X-rays 308, such as but not limited to X-rays in the 1-2 MEV range. In other embodiments, other X-ray 308 dosage may be applied based on the factors such as but not limited to lower X-Ray energy. Other X-ray radiation dosage may include but are not limited to doses in the range of 1 Gray to 60 Grays. When the X-rays 308 encounter the treatment packages 328, the X-rays cause the scintillating matrix and/or polymer to scintillate and the resulting photons strike the release agent, which in turn releases NO 334. The released NO is harmful (deadly) to cells that form the tumor 360 thus providing a non-invasive medically beneficial cancer treatment.

As benefit of the disclosed use of scintillating polymers (or a matrix configuration) as part of the treatment package 328, the X-rays are absorbed at a high efficiency rate by the scintillating polymer, which generates a greater number of photons (light energy), which in turn releases more NO than in the prior art treatment package material and structure.

In one embodiment, the treatment package 328 is formed from at least one or more high atomic weight elements to enhance X-Ray cross section in the organic or inorganic components. In one embodiment, the inorganic component can comprise highly luminescent transition metal clusters with a high concentration of iodine atoms for high average X-Ray cross section. The formulation may be configured with high concentration of iodine atoms to absorb and scatter more X-Rays than can be expected for the organic components. This increases NO release.

One disclosed variant is use of phenyl/methyl silane polymers and copper clusters both known to scintillate in response to X-ray energy. It is disclosed to combine these components into a single material, such as a mixture, layering of materials, or a structure which increase x-ray absorption, photon emissions, and absorption of the same by the NO releasing agents. With this configuration, the scattered secondary X-Rays and Compton electrons can more readily interact with the “bulk” polymer. This mechanism becomes very important as the X-Ray energy is increased to nearly 5 MeV as part of the typical therapeutic dose, which allows for a much higher release of NO in or near the tumor as compared to the prior art.

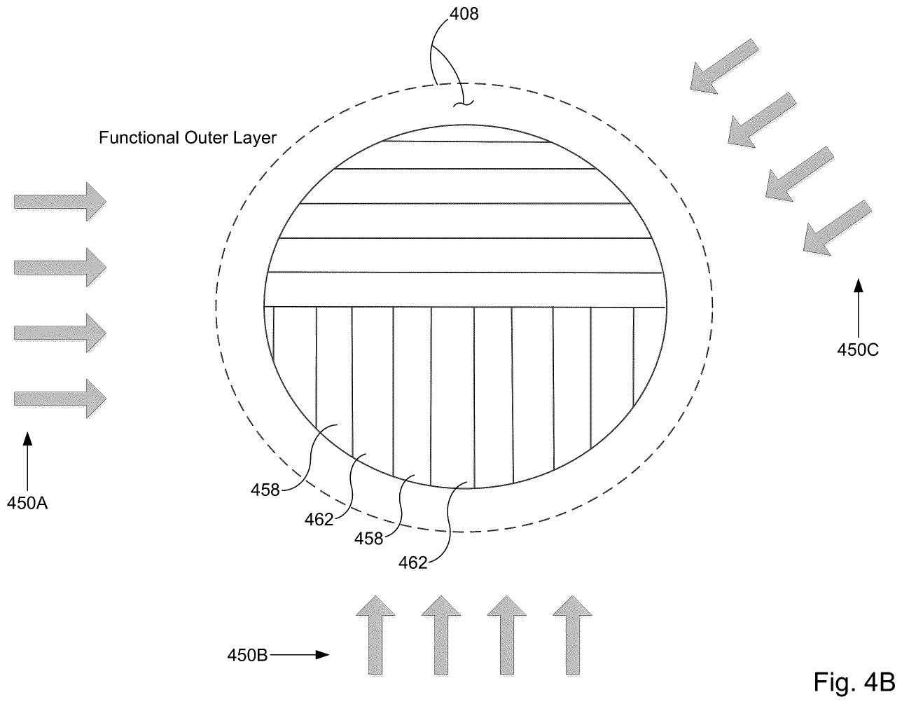

FIG. 4A illustrates an example arrangement of a matrix formed from a scintillating polymer with NO releasing agents embedded therein. This illustrated matrix of scintillating polymer and NO release agent is shown for purposes of discussion and the illustrated inter-connections and structure of the polymer are not to scale nor an accurate depiction of a silicon-based polymer or copolymer.

In this example embodiment, the outer layer 408 comprises a functional material which a macrophage or other similar delivery mechanism will facilitate consumption by a macrophage, thereby resulting in the treatment package 404 being ingested, consumed, attached or otherwise associated with the delivery mechanism. In other embodiments, other functional material types may be provided, such as a functional material which attaches to the macrophage. All, or a portion of the treatment package 404 may be covered in the functional material 408 which is a material that is consumable by a macrophage or likely to be consumed by a macrophage.

In the center area of the treatment package 404 is the polymer and/or matrix 412 which includes, in this embodiment, a polymer that includes scintillating compounds 416 and NO release agents 420. The various concentrations of the polymer and the release agent may be adjusted based on various factors including desired amount of NO production, depth of the tumor being treated, X-ray strength and duration, the type of scintillating material and type of NO release agent, and scintillating output.

FIG. 4B illustrates an alternative arrangement formed by layers of scintillating polymer and NO releasing agents. This is but one possible arrangement of scintillating polymer and NO release agents. In this embodiment, there is an offset layer structure that facilitates a high degree of absorption of the X-ray energy, which in turn increases photon output and the corresponding increase in NO release. As shown in FIG. 4B, the scintillation polymer 458 may be layered with the NO release agent. Various different physical configurations are possible, such that the layers 458, 462 may run in different directions. Other patterns are also contemplated such as but not to limited layers of a checker board pattern, swirls of intermixed material, numerous spheres which gradually shrink in size with alternating layers, or any other pattern. X-ray radiation 450A, 450B, 450C may be directed at the tumor from different directions, which in turn will strike the treatment package 404 at different directions. This provides a benefit of maximizing NO release by maximizing the surface area of scintillating material which are struck by X-rays. In addition, by triangulating or bisecting two or more X-ray beams at the location of the tumor, the dose at the site of the tumor is increased, thereby increasing NO release, while the tissue between an individual X-ray beam and the tumor is exposed to reduced X-ray dose exposure.

By making the entire or majority portion of the treatment package (microsphere) out of a scintillating polymer, the whole microsphere or a majority portion becomes a scintillator. Inside the microsphere is the NO producing compound which is dissolved into the microsphere. The NO producing compound may comprise iron compound, chromium, ruthenium or other compounds which produce NO in response to photons from the scintillating polymer. By using high Z materials, that are heavy elements which absorb X-rays and emit the photons (such as blue light), the amount of photon emission is greatly increased, and the resulting NO production is likewise increased.

Also disclosed is use of sodium gadolinium fluoride nanoparticles due to its high Z number (absorption of X-ray energy). Gadolinium is a high Z (higher atomic number) and as such it absorbs a higher percentage of X-rays than prior art treatment package materials. It is also disclosed that pure or semi-pure copper iodide may be used in the microsphere which forms the treatment package.

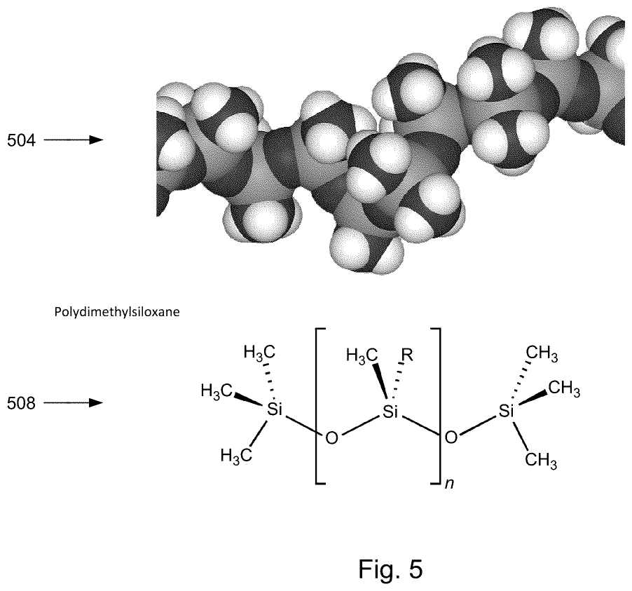

It is also disclosed that the treatment package may be formed from a polymer, such as but not limited to a copolymer configuration. In one embodiment, a silicon polymer is selected as the polymer which is established in a scintillating matrix with NO releasing agents. As shown in FIG. 5, polydimethylsiloxane may be selected as the silicon polymer. As is understood, silicon has four branches, which is to say four bond positions. Silicon in a chain with oxygen is a silane polymer, which may also be referred to as a silane modified polymer. When the silicon bonds methyl groups, the resulting structure is PDMS (Polydimethylsiloxane). These disclosed combinations may be used as or in the scintillating polymer.

FIG. 5 illustrates an example of a silicon-based polymer. The graphic representation 504 of polydimethylsiloxane is illustrated along with the chemical structure 508. This is but one possible silicon-based polymer that may be used in the matrix of the treatment package.

Also disclosed is use of phenyl group (C6H6—putting a benzene in the polymer) or use of naphthalene on the 2-ring group on the silane, instead of the methyl groups, to form an emissive polymer that will scintillate in response to X-rays. The selected copolymer may be referred to as a co-block polymer. In one embodiment, the entire polymer is methyl pendant with phenyl pendant such that it is mostly methyl with less concentrate phenyl. The co-block is or resembles a co-methyl polymer with phenyl groups in it. The co-block is defined by the amount or percent of methyl or phenyl that is mixed in the polymer chain.

It is also contemplated that the composition of the polymer may be tuned by changing the type of molecule or material groups that are put on silicon backbone structure. This type of tuning may occur to place groups on the silicon backbone that generate an emission color from the polymer that corresponds to the absorption of the reagent (such as but not limited to chromium, copper, ruthenium, iron compound) which is placed in the treatment package matrix. As such, depending on the material selected for the NO generator, the polymer may be doped with the molecules that emit light (photons) in a wavelength that maximizes scintillation which in turn will maximize NO production.

Thus, the choice of scintillating polymer and the reagents that are added to the scintillating polymer may be selected to correspond to (align with) the wavelength of the activator of the metal compound that makes and releases NO. By adjusting the materials that form the polymer, NO production may be maximized by adding reagents to shift the wavelength of the emissions to correspond to the absorption of the NO release agent.

In one configuration, the reagents that are added to the polymer are selected to shift the emission of the polymer away from the absorption of the polymer, thereby maximizing allowing a greater percentage of photons to strike and activate the NO producing agent. In one embodiment, the wavelength shift is in the 30 to 40 nm range due to the presence of the phenyl groups. The other goal is to align the polymer emission with the absorption of the NO-releasing compound. This may be greater than 30 nm shift by using an appropriate intermediate wavelength shifting dye or using pendant groups such as naphthalene which shift it further than the phenyl group. Any amount of wavelength shift may be enabled to achieve that emissions from the polymer align with the absorption of the release agent, such as the NO release agent. In one embodiment, the overlap is for Cr (ONO) provides a suitable and well performing relationship. In one embodiment, the shift in emission wavelength is from 350 nm to 450 nm to avoid absorption by a copolymer that absorbs in the 350 nm wavelength range.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A method for reducing a number of cancerous cells in the body comprising:
providing a treatment package having a matrix comprising a mixture of scintillating polymer and a nitric oxide (NO) release agent at least partially surrounded by a functional material;
providing treatment packages to macrophage for consumption by the macrophage causing the treatment package to be within the macrophage;
presenting the macrophage to a human body, the human body having cancer cells living within the human body;
waiting a period of time for the macrophage to travel to the cancer cells;
directing X-ray radiation to the locations of the cancer cells, such that the X-ray radiation cause the scintillating polymer to emit energy at a first wavelength, the energy at a first wavelength causing the NO release agent to release nitric oxide, which reduces the number of cancer cells, wherein the scintillating polymer is an organic silicon-based polymer.

2. The method of claim 1 wherein the matrix is a microsphere.

3. The method of claim 1 wherein the cancer cells form a tumor.

4. The method of claim 1 wherein the scintillating polymer comprises at least polydimethylsiloxane.

5. The method of claim 1 wherein the NO release agent comprises one or more of the following: iron, copper, CrONO, Ruthenium (phen)(NO)(Cl), and Roussin's black salt.

6. The method of claim 1 wherein the scintillating polymer is selected such that the first wavelength of the energy emitted corresponds to an energy wavelength that maximized production of NO from the NO release agent.

7. The method of claim 1 wherein the scintillating polymer and the NO release agent are selected such that an output wavelength of the scintillating polymer aligns with an activation wavelength of the NO release agent.

8. The method of claim 1 wherein the X-ray radiation does is less than 5 MeV.

9. A pharmaceutical treatment package for the delivery of treatment to a cancer tumor cells comprising:
a core comprising a combination of:
a scintillating polymer comprising a polymer which is configured to absorb X-ray energy and in response thereto, emit light energy at a first wavelength when exposed to x-ray radiation;
a release agent which emits a substance that is toxic to cancer tumor cells in response to light energy at the first wavelength; and
a functional coating on at least a portion of an exterior of the core, the functional coating configured to facilitate acceptance or entry of the core into a macrophage.

10. The method of claim 9 wherein the polymer is a silicon polymer matrix.

11. The method of claim 9 wherein the polymer is selected to have 5% or less absorption loss at the first wavelength, wherein the first wavelength is the polymer emission wavelength.

12. The method of claim 9 wherein the 5% or less absorption loss is due to an emissive shift of a pendant phenyl/napthal group.

13. The method of claim 9 wherein the release agent emits nitric oxide.

14. The method of claim 9 wherein the polymer comprises polydimethylsiloxane.

15. The method of claim 9 wherein the release agent is one or more of the following:
inorganic CrONO, Ruthenium (phen)(NO)(Cl), and Roussin's black salt.

16. The method of claim 9 wherein the scintillating polymer is an organic silicone polymer with a pendant group aromaticity with a silane backbone to establish gas permeability.

17. The method of claim 9 wherein the core consists of a scintillating monomers comprising functionalized naphthalene and a release agent which is dispersed in a non-scintillating polymer.
